(19) United States
(12) Patent Application Publication
(10) Pub. No.: US 2022/0079707 A1
(43) Pub. Date: Mar. 17, 2022

(54) METHODS AND DEVICES FOR ACTUATING A CLEARANCE DEVICE FOR CLEARING OBSTRUCTIONS FROM MEDICAL TUBES

(71) Applicant: ClearFlow, Inc., Irvine, CA (US)

(72) Inventors: Kenneth Chesnin, Long Beach, CA (US); Paul Molloy, Irvine, CA (US); Edward M. Boyle, Bend, OR (US); Al Diaz, Irvine, CA (US)

(21) Appl. No.: 17/476,925

(22) Filed: Sep. 16, 2021

Related U.S. Application Data
(60) Provisional application No. 63/079,096, filed on Sep. 16, 2020.

(51) Int. Cl. A61B 90/70; B08B 9/043

(52) U.S. Cl. CPC A61B 90/70; A61B 2090/701; B08B 9/0436

(57) ABSTRACT

A replaceable shuttle for actuating a clearance member. The replaceable shuttle including a shuttle body having an elongated recess formed therein and accessible through a longitudinally-extending, lateral mouth thereof. A magnetic element is disposed within the shuttle body adjacent to the elongated recess. The magnetic element is adapted to be magnetically coupled to a magnetic guide of the clearance member disposed within a tube when such tube is received in the elongated recess through the lateral mouth so that translation of the shuttle body will induce a corresponding translation of the clearance member through the tube.

### BACKGROUND

### Field of the Invention

The invention relates to methods and devices for actuating a clearance device to clear obstructive debris from medical tubes. More particularly, it relates to a removable device having magnetic elements that are magnetically coupled to a clearance member to draw such debris proximally in a medical tube without compromising the sterile field.

### Description of Related Art

Millions of medical tubes are used every year to drain bodily fluids and secretions from within body orifices. For example, such tubes can be used to drain fluid from one's bladder, from the colon or other portions of the alimentary tract, or from the lungs or other organs in conjunction with various therapies. Medical tubes also are used to drain blood and other fluids that typically accumulate within the body cavity following traumatic surgery. In all these cases, a tube is inserted into the patient so that its terminal end is provided in or adjacent the space where it is desired to remove accumulated or pooled fluid, and the proximal end remains outside the patient's body, where it is typically connected to a suction source.

U.S. Pat. No. 7,951,243 (incorporated herein by reference) discloses a clearance device for clearing medical tubes (such as chest tubes) of obstructive clot material. That device relies on a shuttle that is fixed and fitted over a guide tube, to actuate a clearance member within the tube via a magnetic coupling between the shuttle and a magnetic guide linked to the clearance member within the tube. The shuttle disclosed in the '243 patent is fixed over the medical tube and not removable. Thus it must be reproduced with and as part of each unit of the device described in the '243 patent. Moreover, that shuttle is not useful with other magnetically actuated clearance devices as the hospital or other medical facility may have on-hand.

### SUMMARY OF THE INVENTION

A replaceable shuttle for actuating a clearance member. The replaceable shuttle is configured to reversibly engage a tube having a clearance member disposed therein and configured to reversibly engage the clearance member when the shuttle is engaged to the tube.

A replaceable shuttle for actuating a clearance member. The replaceable shuttle including a shuttle body having an elongated recess formed therein and accessible through a longitudinally-extending lateral mouth thereof. The shuttle body is replaceably fitted over and longitudinally translatable along an outer wall of a tube when received in the elongated recess through the lateral mouth. The tube at least partially defines a suction pathway for drawing secretions from a patient. A magnetic element is disposed within the shuttle body adjacent to the elongated recess. The magnetic element is adapted to be magnetically coupled to a magnetic guide of the clearance member through the outer wall of the tube when received in the elongated recess so that translation of the shuttle body along a length of the tube induces a corresponding translation of the clearance member.

A method of clearing obstructions from a tube, including removably positioning a tube within an elongated recess formed in a replaceable shuttle through a longitudinally-extending, lateral mouth thereof; and translating the replaceable shuttle along a length of the tube to correspondingly translate an elongated guide member that is at least partially disposed within the tube and magnetically coupled to the replaceable shuttle.

A device for clearing obstructions from a medical tube, the device including a shuttle guide tube having an inner diameter and an outer diameter. A replaceable shuttle includes a shuttle body having an elongated recess formed therein and accessible through a longitudinally-extending, lateral mouth thereof. The shuttle guide tube being replaceably insertable into and removable from the elongated recess through the lateral mouth. A magnetic element is disposed within the shuttle body adjacent to the elongated recess. An elongated guide member extends through the shuttle guide tube. A clearance member is attached to or formed integrally with the elongated guide member. A magnetic guide is secured to the elongated guide member. The magnetic guide is magnetically coupled to the replaceable shuttle through a wall of the shuttle guide tube so that translation of the replaceable shuttle along a length thereof induces a corresponding translation of the elongated guide member within the shuttle guide tube.

### BRIEF DESCRIPTION OF THE DRAWINGS

FIG. 1 is a schematic perspective illustration showing a clearance device coupled to a medical tube (e.g., a chest tube) that has been placed in a patient recovering from surgery, to permit clearance of the medical tube of obstructions formed therein.

FIG. 2 is a perspective view, partially in section, of a clearance device according to an embodiment hereafter described.

FIG. 3 illustrates an exploded view of a replaceable shuttle for actuating the clearance device of FIGS. 1 and 2.

FIG. 4A illustrates a perspective view of the replaceable shuttle of FIG. 3 reversibly fitted over a guide tube.

FIG. 4B illustrates the process of fitting the replaceable shuttle of FIG. 3 over the guide tube.

FIG. 5 illustrates a top view of the replaceable shuttle of FIG. 3.

FIG. 6 illustrates a front view of the replaceable shuttle of FIG. 3.

FIG. 7 illustrates a side view of the replaceable shuttle of FIG. 3.

FIG. 8 illustrates a rear view of the replaceable shuttle of FIG. 3.

FIGS. 9A-9C are perspective views of a clearance device coupled to a chest tube schematically showing the replaceable shuttle, and correspondingly the guide wire and clearance member, at different stages of advancement for clearing obstructions from the chest tube.

### DETAILED DESCRIPTION OF PREFERRED EMBODIMENTS

As used herein, the terms proximal and distal are generally to be construed with reference to a patient that has been or is to be fitted with a medical tube, such as a chest tube. For example, the distal end or region of a medical tube (e.g., a chest tube) is that end or region that is to be inserted into or disposed more adjacent (e.g., within) the patient during use, as compared to the opposite end or region of the medical tube (e.g., chest tube). Similarly, a distal element (or the distal side or region of an element) is nearer to the patient, or to the distal end of the chest tube, than a proximal element (or the proximal side or region of an element). Also herein, the “terminal” end of a tube, wire or member refers to its distal end.

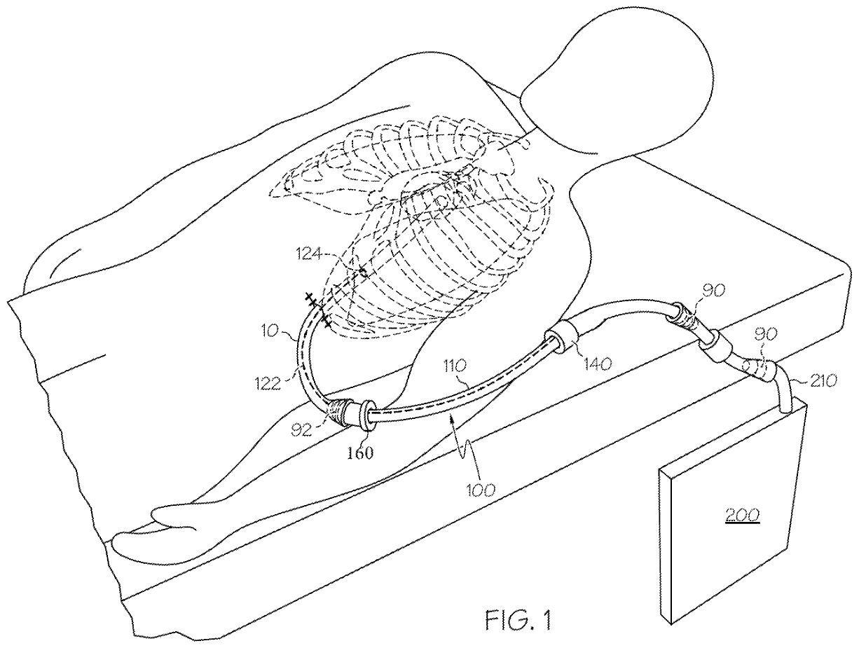

FIG. 1 shows a schematic representation of a medical tube being used to drain accumulated fluid from within the body cavity of a patient, in accordance with an exemplary embodiment of the invention. In FIG. 1 the medical tube is inserted into and used to drain fluid from the chest cavity of the patient, and so is a chest tube 10 as described in the '243 patent incorporated above. The remaining description will be provided with reference to chest tubes 10. However, the body tube may be one used in other applications as also described in the '243 patent.

Returning to FIG. 1, the chest tube 10 enters the patient through the chest-cavity (body) wall, so that its distal end is positioned within the chest (body) at a location from which fluid is to be drained. The proximal end of the chest tube 10 remains outside the body. The chest tube 10 can be inserted into the patient in a conventional manner and positioned and secured in place through the chest-cavity wall by the physician. A clearance device 100 is fitted to the proximal end of the chest tube 10. The clearance device 100 includes a shuttle guide tube 110 (described below) that is connected to the proximal end of the chest tube 10 and is provided in fluid communication therewith. The clearance device 100 also includes a clearance member 124 that can be reversibly advanced into and through the chest tube 10 to withdraw obstructive debris therefrom (also described below). The proximal end of the shuttle guide tube 110 (i.e., the end opposite the point of connection to the chest tube 10) is connected to a suction source 200, e.g., via a vacuum tube 210. The suction source draws a suction within the chest tube 10, via the shuttle guide tube 110 and vacuum tube 210 (if present), both to draw fluid out of the body cavity and to sustain the normal physiologic negative pressure within the chest.

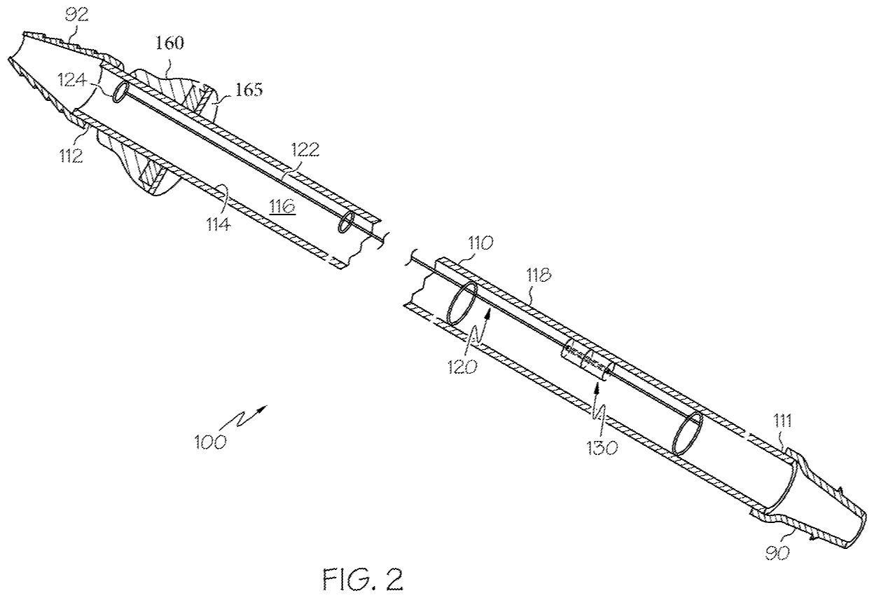

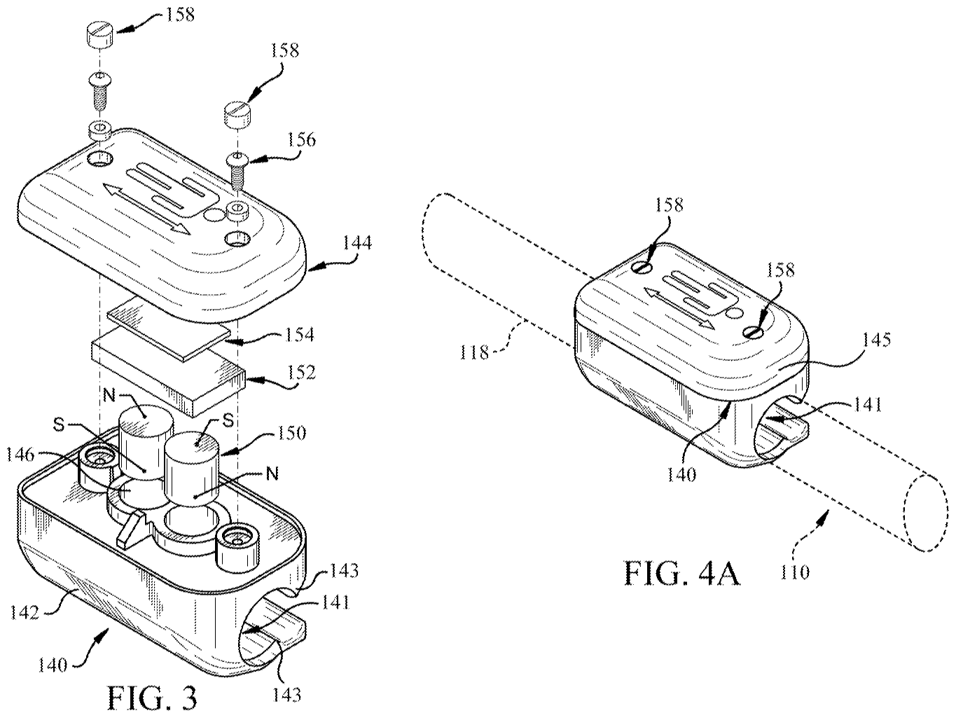

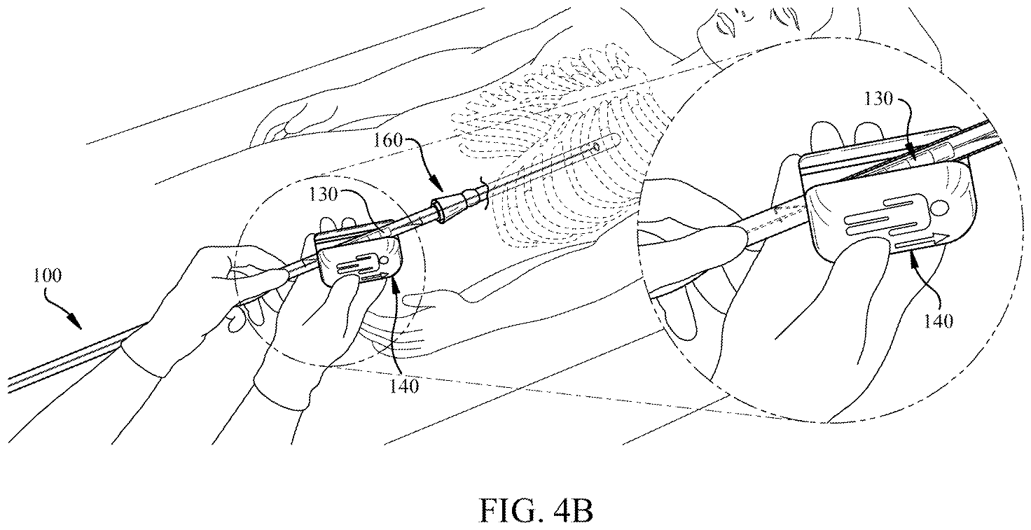

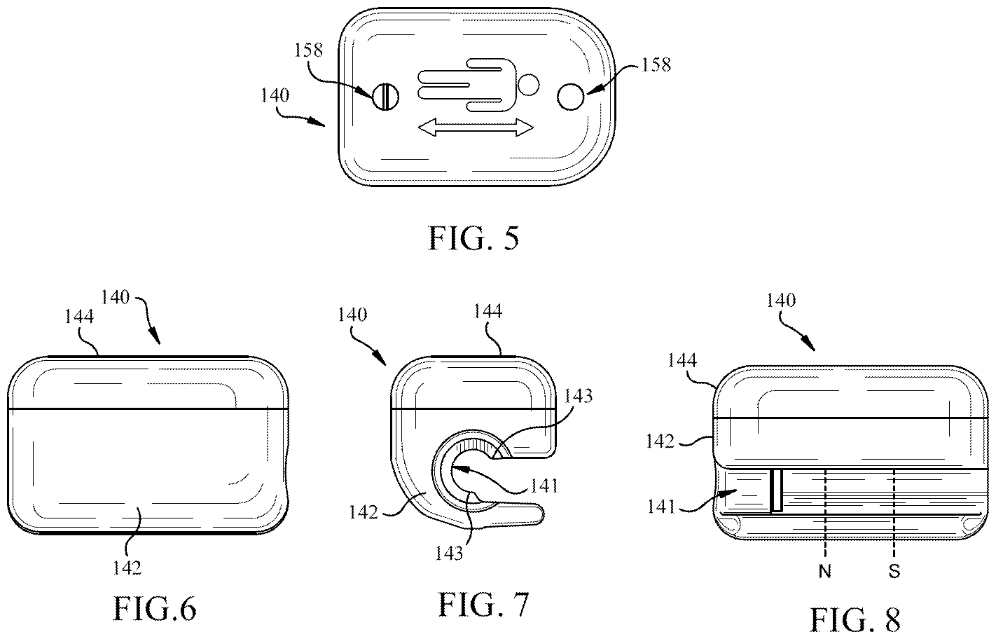

Exemplary embodiments of the clearance device 100 will now be more fully described. As seen in FIG. 2, the clearance device 100 includes the shuttle guide tube 110 mentioned above. The shuttle guide tube 110 has a proximal end 111 and a distal end 112. In use, the proximal end 111 of the shuttle guide tube 110 is adapted to be connected to a suction source, preferably via a suction fitting 90 secured to its proximal end, and the distal end 112 is adapted to be connected to a medical tube, such as the chest tube 10, preferably via a chest-tube fitting 92, secured to its distal end. Guide tube 110 has a wall having an inner diameter 114 defining a guide-tube passageway 116 and an outer circumference 118. A removable and replaceable shuttle 140 (FIG. 4A) may be selectively fitted over the guide tube 110 at its outer circumference 118 and is adapted to translate along the length of the tube 110 to advance and withdraw the clearance member 124 as described in detail below. In FIG. 1 the replaceable shuttle 140 is schematically represented. FIGS. 3-8 (described in detail below) illustrate structural details of the replaceable shuttle 140.

A wire clearance assembly 120 is at least partially disposed within the guide-tube passageway 116 as explained in the '243 patent. Briefly, the wire clearance assembly 120 includes an elongate guide member 122 and a clearance member 124 disposed in and secured to the distal region of the guide member 122, preferably at its distal end. Optionally, the guide member 122 can be in the form of a guide wire, and the clearance member 124 can be formed by the guide wire, e.g., as a loop.

A magnetic guide 130 (e.g., permanent magnets) is secured to the guide member 122 in the proximal region thereof, again as described in the '243 patent incorporated herein.

As noted above and illustrated in FIGS. 4A and 4B, the replaceable shuttle 140 may be selectively fitted over the outer circumference 118 of the guide tube 110. As illustrated in FIG. 4B, a user can place the replaceable shuttle 140 on the guide tube 110 at a location proximate the magnetic guide 130 within the guide tube 110. Alternatively, the replaceable shuttle 140 may be fitted in a different location and then slid along the guide tube 110 until it reaches the location of the magnetic guide 130. So long as the magnetic guide 130 is constrained from moving away from the shuttle (e.g., if its translation away from the shuttle is prevented because the guide member is fully inserted or fully withdrawn against a stop), the replaceable shuttle 140 can be advanced over the magnetic guide 130 until it magnetically engages the magnetic guide 130 through the tube 110 wall.

Referring to FIGS. 3, 4A-B and 8, the replaceable shuttle 140 has an elongated recess 141 preferably having a curved inner surface that is complementary and substantially corresponds to the outer perimeter shape of the guide tube 110, or in the case of a cylindrical tube, its outer circumference 118. To allow longitudinal translation of the replaceable shuttle 140, the diameter of the elongated recess 141 approximates or is greater than the outer diameter of the guide tube 110. To ensure lateral retention of the replaceable shuttle 140 over the guide tube 110 in-use, the recess 141 preferably includes at least one projection 143 that will interfere with lateral (as opposed to longitudinal or revolutionary—i.e., about the tube) movement of the replaceable shuttle 140 relative to the tube 110. The projection 143 can be a tab, a bulge, a raised surface, a protuberance or any other structure that creates a localized reduction in the distance between opposing surfaces or portions of the inner surface of the elongated recess 141. In the embodiment illustrated, the elongated recess 141 is generally C-shaped, having a substantially continuous C-shaped inner surface to complement and accommodate a cylindrical guide tube 110 therein, with opposing projections 143 extending toward one another at a longitudinally-extending, lateral mouth of the elongated recess 141 so as to inhibit removal of the replaceable shuttle 140 from the tube 110. The surface defining the elongated recess 141 may be smooth or it may have raised bearing pads or surfaces for reducing the surface-to-surface contact between the replaceable shuttle 140 and the guide tube 110. The reduced surface-to-surface contact may reduce the friction between the replaceable shuttle 140 and the guide tube 110 to aid in longitudinally translating the replaceable shuttle 140 along the guide tube 110.

The elongated recess 141 is contoured and dimensioned such that the replaceable shuttle 140 can be fitted over the guide tube 110 and be slidably and smoothly translated along the length of the guide tube 110 with the guide tube 110 received in the recess 141 (e.g., within or against its inner surface). It is contemplated that the replaceable shuttle 140 may be retained on the guide tube 110 in a snap-fit manner. For example, a single projection 143 may extend from one side of the lateral mouth of the elongated recess 141, thus effectively constraining the height of that mouth to a reduced-height gap. The single projection 143 may be dimensioned such that a gap between it and the opposing surface (or portion of the inner surface of the recess) is less than the outer diameter of the guide tube 110. As such, during insertion or removal of the guide tube 110 into or out of the elongated recess 141 through the lateral mouth thereof, the guide tube 110 will be deflected by the projection 143 as it passes the aforementioned gap so that it deforms to accommodate the reduced height of the gap. Once the guide tube 110 passes the gap and is either fully received in the elongated recess 141, or fully removed through the mouth thereof, the tube elastically returns to its unconstrained, original shape. In the embodiment shown, the replaceable shuttle 140 includes opposing projections 143. It is contemplated that there may be only a single projection, or a plurality of spaced-apart projections along the length of the elongated recess 141, or a continuous projection that extends the entire length of the elongated recess 141.

Optionally, when the guide tube is made from a more rigid material and thus less amenable to being compressed on traversing the gap, one or both of projections 143 can be deflectable on insertion of the guide tube 110 therethrough, so as to accommodate passage of the guide tube 110 through the lateral mouth and into the elongated recess 141. Upon seating the guide tube 110 therein, the deflected projection(s) 143 return(s) to its/their resting configuration, thus laterally retaining the guide tube 110 within the elongated recess 141. To remove that guide tube 110 from the elongated recess 141, it is drawn laterally, against the bias of the projection(s) 143 to allow passage of the guide tube 110 through the lateral mouth, this time out from the elongated recess 141 to separate the replaceable shuttle 140 from the guide tube 110. In this manner, the replaceable shuttle 140 is replaceable on and over the guide tube 110, or successive or multiple such guide tubes when or as needed.

It is also contemplated that other methods can be used to retain the replaceable shuttle 140 on the guide tube 110, such as, but not limited to, an interference fit.

Referring to FIG. 3, the replaceable shuttle 140 includes a body 142 and a cover 144. Fasteners 156 are provided for securing the cover 144 to the body 142 of the replaceable shuttle 140. Plugs 158 may be provided for covering the fasteners 156 and for providing a smooth external appearance. Optionally, the cover and body can be assembled without fasteners using methods known in the state of the art such as ultrasonic welding, adhesive binding, laser welding or other methods. Two recesses 146 are formed in an upper surface of the body 142 and are dimensioned to receive two magnetic elements 150. In the illustrated embodiment, the magnetic elements 150 are provided in the form of cylindrical plugs that are received into mating recesses 146. When they are provided in the form of permanent magnets, the magnetic elements 150 are oriented such that their respective North and South poles face opposite directions. In other words, the North pole of one magnetic element 150 faces the elongated recess 141 while the South pole of the other magnetic element 150 faces the elongated recess 141. This results in the two magnets 150 creating a single North pole and a single South pole in the elongated recess 141 (see FIG. 8). Optionally, a single magnetic element or a series of individual such elements (not shown) can be aligned along and parallel to the elongated recess 141, thereby defining overall magnetic North and South poles axially spaced along that recess 141 at locations corresponding to the counterpart South and North magnetic poles of the magnetic guide 130 within the tube 110. Optionally, a single horseshoe magnet could be provided with its poles directed similarly as discussed above and shown in FIG. 3.

As with the magnetic guide 130 discussed above, the magnetic elements 150 can be permanent magnets or, optionally, metal elements having magnetic properties that are not necessarily permanent magnets. However, for reasons that will become clear, either at least the magnetic guide 130 or at least one of the magnetic elements 150 should be a permanent magnet. Optionally, both the magnetic guide 130 and the magnetic elements 150 are permanent magnets.

Optionally, a magnetic shield 152 can be positioned adjacent or over the magnetic elements 150. Referring to FIG. 3, a foam strip or adhesive tape 154 may be used to secure the magnetic shield 152 in the replaceable shuttle 140 and/or prevent the magnetic shield 152 and magnetic elements 150 from freely moving or rattling within the replaceable shuttle 140. Depending on the magnetic strength of the magnetic elements 150, such a shield 152 may be desirable to prevent a strong magnetic field from interfering with medical equipment in close proximity with the clearance device 100 (e.g., an implanted pacemaker). While the shield 152 cannot completely enclose the magnetic elements 150 and the magnetic guide 130 within the tube 110 (e.g., the tube 110 passes through the recess 141 of the replaceable shuttle 140 and the magnetic guide 130 and elements 150 are positioned radially within as they interact with one another), such a shield 152 can help to reduce the magnetic field that extends beyond the replaceable shuttle 140. The magnetic shield 152 can be in direct contact with the magnetic elements as shown in FIG. 3. By connecting opposite poles of the magnetic elements 150 using a material with high magnetic permeability such as low carbon steel, MuMETAL® (Magnetic Shield Corporation, Bensenville, Ill.) or other materials known in the art, the shield also serves to direct the magnetic field such that greater magnetic attraction to magnetic elements 150 is achieved.

When the magnetic guide 130 and the magnetic elements 150 are magnetically coupled, they all will be disposed within the volume of the replaceable shuttle 140. It is contemplated that the magnetic shield may extend circumferentially around the elongated recess 141 to surround the coupled magnetic guide 130 and magnetic elements 150, except for the lateral mouth through which the tube is inserted into the elongated recess 141. Optionally, the magnetic guide 130 may be provided as metal elements that are not permanent magnets, or as relatively weak permanent magnets, so as not to create strong magnetic fields that may interfere with other equipment when the replaceable shuttle 140 is removed from the guide tube 110. The magnetic guide 130 and the magnetic elements 150 may have a residual flux density (Br) of, e.g., 14-15 kGs, such as 14.3 to 14.8 kGs.

When provided as permanent magnets, the North pole of the magnetic guide 130 aligns with the South pole of the magnetic element(s) 150 in the replaceable shuttle 140, and the South pole of the magnetic guide 130 aligns with the North pole of those magnetic element(s) 150 when the replaceable shuttle 140 is fitted over the guide tube 110, located in its recess 141. The selection of particular magnets, having appropriate magnetic strength, is well within the capability of a person having ordinary skill in the art. Optionally, the magnetic guide 130 and magnetic element(s) 150, and their cooperative attractive strengths, are selected to allow a high degree of attractive force to prevent as much as possible instances of magnetic de-coupling between the magnetic guide 130 and the replaceable shuttle 140, while at the same time minimizing their weight and bulk.

A shuttle stop 160 is secured to the outer circumference 118 of the guide tube 110 in a distal region thereof, preferably just proximal to the distal end of the guide tube 110. The replaceable shuttle 140 and shuttle stop 160 can have complementary first and second surfaces 145 (FIG. 4A) and 165 (FIG. 2), which face one another. As the replaceable shuttle 140 is translated distally along the length of the guide tube 110, it approaches and ultimately reaches a position wherein the respective surfaces 145 and 165 are in contact or disposed adjacent one another.

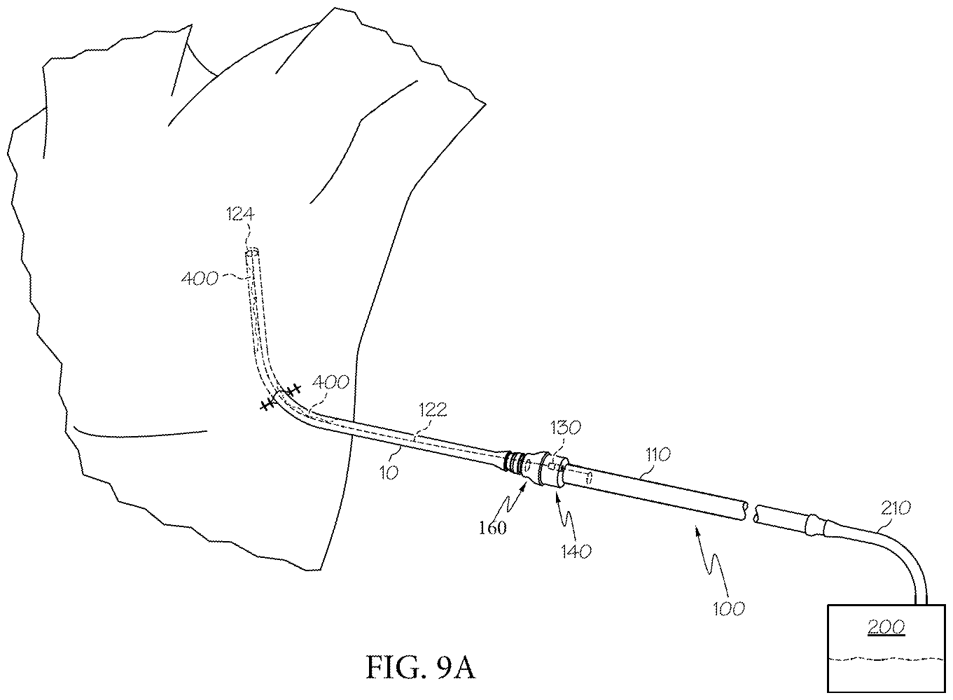

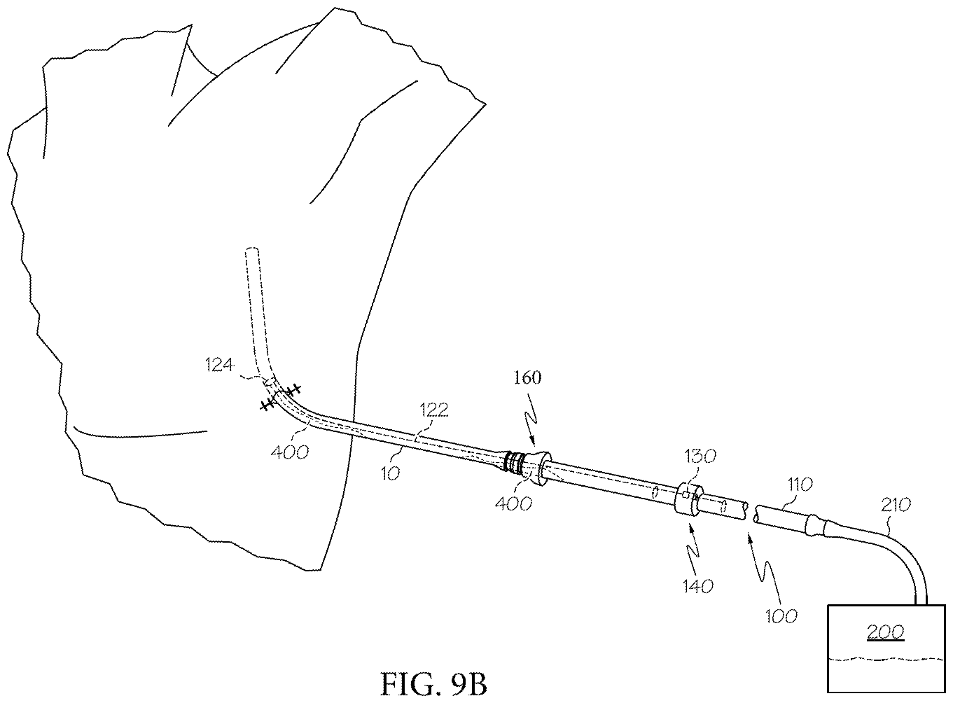

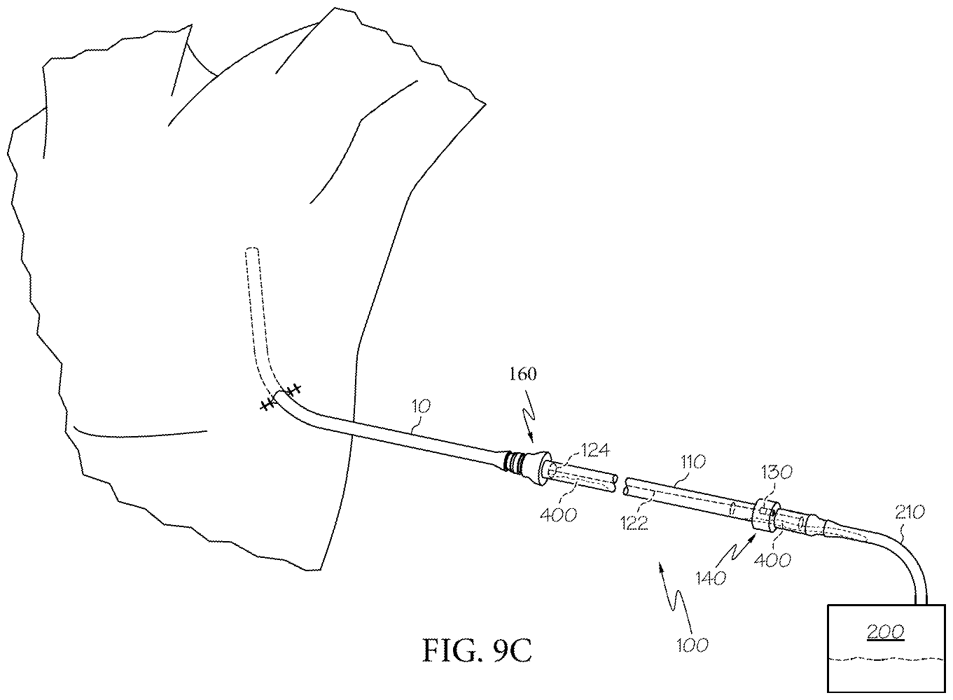

Referring now to FIGS. 9A-9C, the clearance device 100 described above is shown fitted to a chest tube 10. The chest tube 10 has a wall having an outer circumference and an inner diameter that defines a chest-tube passageway. The clearance device 100 is fitted to the chest tube 10 via a chest-tube fitting 92 that ensures a fluid-tight connection between the distal end of the shuttle guide tube 110 and the proximal end of the chest tube 10, while providing fluid communication between the chest-tube passageway and the guide-tube passageway 116.

With the clearance device 100 and chest tube 10 fitted together as described above, the guide member 122 and the clearance member 124 disposed at its distal end may be advanced into and withdrawn from the chest tube 10 to assist in clearing debris therefrom as follows. In use, the magnetic guide 130 and the magnetic elements 150 of the replaceable shuttle 140 are magnetically attracted and coupled to one another when the replaceable shuttle 140 is fitted over the guide tube 110. This results in coupling the magnetic guide 130 to the replaceable shuttle 140 via magnetic forces that act through the guide tube 110 wall. Consequently, longitudinally sliding or translating the replaceable shuttle 140 along the length of the shuttle guide tube 110 induces a corresponding translational movement of the magnetic guide 130 magnetically coupled thereto, and of the guide member 122 that is secured to the magnetic guide 130. In FIG. 9A, the replaceable shuttle 140 (shown schematically in this figure) is illustrated in a first position, in contact with the shuttle stop 160. The length of the guide member 122 between its distal end and the point where it is secured to the magnetic guide 130 is preferably selected to be substantially equal to the length of the chest tube 10 plus the length corresponding to the distance between the shuttle stop 160 and the point where the chest tube 10 engages the fitting 92. Optionally, when the replaceable shuttle 140 is positioned against the shuttle stop 160 (having the magnetic guide 130 in tandem therewith along the guide-tube 110 length), the clearance member 124 at the distal end of the guide member 122 is disposed within the chest tube 10 adjacent its distal end and does not emerge from the chest tube 10 into the body cavity. Optionally, this is the first position of the clearance member 124, where it normally rests when the clearance device 100 is not being used to actively remove debris from the chest tube 10.

In operation, with the distal end of the chest tube 10 inserted in a body cavity of a patient and the shuttle guide tube 110 being connected to a suction source 200 at its proximal end, fluid from the body cavity is drawn into and through the chest-tube passageway, then through the guide-tube passageway 116 to be collected or disposed of in any suitable or conventional manner, such as in a conventional collection canister (not shown). Optionally, the clearance member 124 is in the form of a wire loop that scrapes the inner diameter of the chest tube 10 as it translates along the chest-tube 10 length.

As noted above, the clearance member 124 (e.g., loop) is normally disposed adjacent the distal end of the chest tube 10 inside the chest-tube passageway. To help clear the chest tube 10 of clots and other debris 400 accumulated therein, a nurse, physician, or other operator places the replaceable shuttle 140 on the tube 110 (FIG. 9A) so that it is fitted over the tube 110, which is received within the recess 141, e.g., via the snap-fit process described above. When so fitted, and once it is magnetically coupled with the magnetic guide 130 within the tube 110, she then pulls the replaceable shuttle 140 proximally along the length of the guide tube 110, toward the tube's 110 proximal end. The attractive magnetic force between the magnetic guide 130 and the magnetic elements 150 retains the magnetic guide 130 in tandem with the replaceable shuttle 140 as the latter translates proximally, which in turn draws the guide member 122 and clearance member 124 proximally through the chest-tube passageway as seen in FIG. 9B. As the clearance member 124 is drawn proximally, it engages clot material and other debris in its path and forces such material and debris proximally (FIGS. 9B and 9C), toward the proximal end of the chest-tube passageway and ultimately out of that passageway, and into the guide-tube passageway 116 (FIG. 9C). To carry out this operation, preferably the operator grasps the replaceable shuttle 140 with one hand and the proximal end of the guide tube 110 with the other hand so that the pulling force applied to the replaceable shuttle 140 is applied against a counterforce applied to the tube 110 via the other hand, and not against sutures retaining the chest tube 10 in place in the patient. Optionally, the same objective can be achieved by grasping a different portion of the guide tube 110, or the shuttle stop 160, with the other hand before sliding the replaceable shuttle 140. Optionally, the clearance member can be alternately withdrawn and advanced from/into the chest-tube passageway to help break up clot material or other debris, as well as to aid in drawing such debris proximally. Once the clearance operation has ended, the replaceable shuttle 140 may be used to restore the magnetic guide 130, and consequently the clearance member 124, to its resting position, and then the replaceable shuttle 140 can be removed from the tube 110 and stored for later use—or used elsewhere where the replaceable shuttle 140 is needed; e.g., to actuate a different clearance device associated with a different chest tube, surgical drain or other body tube—in the same patient or in a different patient in the same facility.

As will be appreciated, while the replaceable shuttle 140 is being used to actuate a clearance member 124 within a medical tube, if it becomes de-coupled from the magnetic guide 130 within the guide tube 110, the replaceable shuttle 140 and the magnetic guide 130 may be magnetically re-coupled by advancing the replaceable shuttle 140 forward until magnetic coupling is re-established. Alternatively, the operator may squeeze the chest tube 10 or guide tube 110 to manually engage the guide member 122 through the tube wall and hold it in position while the replaceable shuttle 140 is translated to magnetically re-engage the magnetic guide 130 through the guide-tube 110 wall.

Although the invention has been described with respect to certain preferred embodiments, it is to be understood that the invention is not limited by the embodiments herein disclosed, which are exemplary and not limiting in nature, but is to include all modifications and adaptations thereto as would occur to the person having ordinary skill in the art upon reviewing the present disclosure, and as fall within the spirit and the scope of the invention as set forth in the appended claims.

What is claimed is:

1. A replaceable shuttle for actuating a clearance member, comprising a replaceable shuttle configured to reversibly engage a tube having a clearance member disposed therein and configured to reversibly engage the clearance member when the shuttle is engaged to the tube.

2. The replaceable shuttle of claim 1, further comprising:
a shuttle body having an elongated recess formed therein and accessible through a longitudinally-extending, lateral mouth thereof; and
a magnetic element disposed within said shuttle body adjacent to said elongated recess, said magnetic element being adapted to be magnetically coupled to a magnetic guide of the clearance member disposed within the tube when the tube is received in said elongated recess through said lateral mouth so that translation of said shuttle body will induce a corresponding translation of said clearance member through said tube.

3. The replaceable shuttle of claim 2, the magnetic element being selected from the group consisting of metal elements having magnetic properties and permanent magnets.

4. The replaceable shuttle of claim 2, said elongated recess being C-shaped.

5. The replaceable shuttle of claim 2, further comprising a projection formed at said lateral mouth thereby defining a reduced-height gap between said projection and an opposing surface relative to a height of said lateral mouth.

6. The replaceable shuttle of claim 5, said elongated recess being C-shaped, said reduced-height gap being less than a diameter of an inner surface of the C-shaped elongated recess.

7. A replaceable shuttle for actuating a clearance member, the replaceable shuttle comprising:
a shuttle body having an elongated recess formed therein and accessible through a longitudinally-extending, lateral mouth thereof, said shuttle body being replaceably fitted over and longitudinally translatable along an outer wall of a tube when received in said elongated recess through said lateral mouth, said tube at least partially defining a suction pathway for drawing secretions from a patient; and
a magnetic element disposed within said shuttle body adjacent to said elongated recess, said magnetic element being adapted to be magnetically coupled to a magnetic guide of the clearance member through the outer wall of said tube when said tube is received in said elongated recess so that translation of said shuttle body along a length of said tube induces a corresponding translation of said clearance member.

8. The replaceable shuttle of claim 7, said elongated recess being C-shaped.

9. The replaceable shuttle of claim 8, further comprising a projection formed at the lateral mouth of the elongated recess wherein a gap defined between the projection and an opposing surface is less than a diameter of an inner wall of the C-shaped elongated recess.

10. The replaceable shuttle of claim 7, said replaceable shuttle being fitted over said tube received within said elongated recess in a snap-fit manner.

11. The replaceable shuttle of claim 7, the magnetic element being selected from the group consisting of metal elements having magnetic properties and permanent magnets.

12. A method of clearing obstructions, comprising removably positioning a tube within an elongated recess formed in a replaceable shuttle through a longitudinally-extending, lateral mouth thereof; and translating the replaceable shuttle along a length of the tube to correspondingly translate an elongated guide member that is at least partially disposed within said tube and magnetically coupled to said replaceable shuttle.

13. The method of claim 12, a magnetic guide being secured to said elongated guide member in a proximal region thereof, said replaceable shuttle having a magnetic element, said elongated guide member and said replaceable shuttle being magnetically coupled through cooperation of said magnetic guide and said magnetic element.

14. The method of claim 13, said magnetic element being selected from the group consisting of metal elements having magnetic properties and permanent magnets.

15. The method of claim 12, wherein translation of said replaceable shuttle and a corresponding translation of said elongated guide member do not compromise a sterile field at least partially defined within said tube.

16. The method of claim 12, said replaceable shuttle being secured to the tube in a snap-fit manner.

17. The method of claim 16, said elongated recess being C-shaped.

18. The method of claim 17, said replaceable shuttle comprising a projection formed at the lateral mouth of the elongated recess wherein a gap formed between the projection and an opposing surface is less than a diameter of an inner surface of the C-shaped elongated recess.

19. A device for clearing obstructions from a medical tube, the device comprising:
a shuttle guide tube having an inner diameter and an outer diameter,
a replaceable shuttle comprising:
a shuttle body having an elongated recess formed therein and accessible through a longitudinally-extending, lateral mouth thereof, said shuttle guide tube being replaceably insertable into and removable from said elongated recess through said lateral mouth; and
a magnetic element disposed within said shuttle body adjacent to said elongated recess,
an elongated guide member extending through said shuttle guide tube,
a clearance member attached to or formed integrally with said elongated guide member, and
a magnetic guide secured to said elongated guide member, said magnetic guide being magnetically coupled to said replaceable shuttle through a wall of said shuttle guide tube so that translation of said replaceable shuttle along a length thereof induces a corresponding translation of said elongated guide member within said shuttle guide tube.

20. The device of claim 19, further comprising a projection formed at said lateral mouth thereby defining a reduced-height gap between said projection and an opposing surface of said shuttle body relative to a height of said lateral mouth.

21. The device of claim 19, said elongated recess being C-shaped, said gap being less than the outer diameter of said shuttle guide tube when the shuttle guide tube is uncompressed.
